(19) United States
(12) Patent Application Publication
(10) Pub. No.: US 2019/0117992 A1
(43) Pub. Date: Apr. 25, 2019

(54) PHOTOTHERAPY APPARATUS

(71) Applicant: SHARP KABUSHIKI KAISHA, Sakai City, Osaka (JP)

(72) Inventors: KATSUJI IGUCHI, Sakai City (JP); HIROYA SATO, Sakai City (JP); TAKASHI YOSHIMOTO, Sakai City (JP); JUN MORI, Sakai City (JP)

(73) Assignee: SHARP KABUSHIKI KAISHA, Sakai City, Osaka (JP)

(21) Appl. No.: 16/309,091

(22) PCT Filed: Jun. 26, 2017
(86) PCT No.: PCT/JP2017/023336

§ 371 Date: Dec. 11, 2018

(30) Foreign Application Priority Data
Jun. 30, 2016 (JP) 2016-130941

(51) Int. Cl. A61N 5/06

(52) U.S. Cl. CPC A61N 5/06; A61N 2005/002

(57) ABSTRACT

A phototherapy apparatus includes: a light irradiation module that radiates light for treatment; and a spacer that is disposed on a light output side of the light irradiation module and contacts a surface of a body to keep a distance between the light irradiation module and a part to be treated on the surface of the body constant, in which a spacer opening that avoids contact of the part to be treated and the spacer is formed on a surface of the spacer, which contacts the surface of the body.

### TECHNICAL FIELD

The present invention relates to a phototherapy apparatus used for light irradiation therapy.

### BACKGROUND ART

Light irradiation is utilized for various purposes including treatment for disease such as neonatal jaundice, psoriasis, or acne, alleviation of pain., and cosmetics. Specifically, green light and blue-white light are used for treatment for neonatal jaundice, ultraviolet light is used for treatment for psoriasis, and blue light, red light, and yellow light are used for treatment for acne. In this manner, various light sources are used in accordance with intended use.

For example, in a case of a light source such as an excimer lamp or an arc lamp, therapeutic light is radiated to an affected part arranged at a constant distance from the fixed light source. However, in a case where such a light source of a lamp type is used, an irradiation area is too large and a part other than an affected part is also irradiated with therapeutic light, so that there is a concern about various side effects on a normal part. Thus, some shielding countermeasures need to be taken to prevent the irradiation to the normal part with therapeutic light, and treatment takes time and effort.

For example, in a case where disease developed in a part of a face is treated, a mask for eyes (blindfold) with which eyes that are normal parts are protected is necessary, and, furthermore, a mask which exposes only an affected part of the face is also necessary to protect normal parts of the face. Moreover, for the treatment, a patient is required to keep his/her posture almost without moving for several tens of minutes in a state where his/her body is restrained, and such an experience is not pleasant even for the treatment.

In a case where an affected part has a curved surface, for example, like an arm or a foot, depending on a part such as a front part, rear part, or a side part, an irradiating apparatus of a lamp type may force a patient to take an unnatural posture. In addition, irradiation intensity is different for each position of the affected part depending on an angle or a distance of the affected part having the curved part with respect to the lamp, so that it is difficult to irradiate an entirety of the affected part with uniform therapeutic light in some cases. Further, the apparatus using such a light source of the lamp type has many accompanying devices such as a power source and a cooling device and is large-sized, so that a large space is required for installation and a price of the apparatus becomes high.

Under such circumstances, some techniques by which light irradiation is able to be performed with an affected part directly covered have been proposed. For example, PTL 1 discloses a phototherapy apparatus that has flexibility and performs light irradiation by winding an optical pad, in which a large number of LEDs serving as light sources are arranged on a film substrate, around an affected part. PTL 2 discloses a phototherapy apparatus in which an LED serving as a light source is arranged on a film substrate and a light-transmitting material is held between an affected part and the LED so that light emitted from the LED is able to be transmitted to the affected part.

### CITATION LIST

### Patent Literature

PTL 1: International Publication. No. WO2001014012A1 (published on Mar. 1, 2001)

PTL 2: International Publication No. WO2012023086A1 (published on Feb. 23, 2012)

### SUMMARY OF INVENTION

### Technical Problem

However, related arts described above have a problem that it is difficult to perform treatment without directly contacting an affected part.

That is, in a configuration of PTL 1 or 2, the optical pad or the light-transmitting material directly contacts an affected part in treatment. Thus, when the affected part is ulcerous or raised, a patient feels a pain or the affected part is pressurized.

The invention is made in view of the aforementioned problems and an object thereof is to provide a phototherapy apparatus that performs light irradiation by directly covering as affected part and is capable of performing treatment without directly contacting the affected part that is a part to be treated.

### Solution to Problem

In order to solve the aforementioned problems, a phototherapy apparatus according to an aspect of the invention includes: a light irradiation module that radiates light for treatment; and a spacer that is disposed on a light output side of the light irradiation module and contacts a surface of a body to keep a distance between the light irradiation module and a part to be treated on the surface of the body constant, in which an opening that avoids contact of the part to be treated and the spacer is formed on a surface of the spacer, which contacts the surface of the body.

### Advantageous Effects of Invention

According to an aspect of the invention, an effect that a phototherapy apparatus capable of performing treatment without directly contacting an affected part that is a part to be treated is able to be provided is exerted.

### BRIEF DESCRIPTION OF DRAWINGS

FIG. 1 is a bird's eye view illustrating a state where a phototherapy apparatus according to Embodiment 1 is mounted on a body.

FIG. 2 is a schematic sectional view illustrating the state where the phototherapy apparatus is mounted on the body.

FIG. 3 is a plan view illustrating the state where the phototherapy apparatus is mounted on the body.

FIG. 4 is a sectional view of an apparatus main body of the phototherapy apparatus.

FIG. 5 is a plan view of the light irradiation module.

FIG. 6 is a plan view of a back surface of the light irradiation module illustrated in FIG. 5.

FIG. 7(a) is a plan view of a spacer of a phototherapy apparatus according to Embodiment 2 and FIG. 7(b) is a sectional view of the spacer illustrated in FIG. 7(a).

FIG. 8 is a plan view illustrating a state where a spacer opening is provided in the spacer illustrated in FIG. 7.

FIG. 9 is a plan view of a light irradiation module of a phototherapy apparatus according to Embodiment 3.

FIG. 10 is a plan view of a back surface of the light irradiation module illustrated in FIG. 9.

FIG. 11 is a plan view of a spacer of a phototherapy apparatus according to Embodiment 4.

FIG. 12 is a sectional view of an apparatus main body of the phototherapy apparatus according to Embodiment 4.

FIG. 13 is a sectional view of an apparatus main body of a phototherapy apparatus according to Embodiment 5.

FIG. 14 is a sectional view of an apparatus main body of a phototherapy apparatus according to Embodiment 6.

FIG. 15 is a sectional view of an apparatus main body of a modified example of the phototherapy apparatus illustrated in FIG. 14.

FIG. 16 is a sectional view of an apparatus main body of a phototherapy apparatus according to Embodiment 7.

FIG. 17 is a sectional view of an apparatus main body of a modified example of the phototherapy apparatus illustrated in FIG. 16.

### DESCRIPTION OF EMBODIMENTS

Embodiments of the invention will be described in detail below. Note that, for convenience of description, a member having the same function as that of a member indicated in each embodiment will be given the same reference sign and description thereof will be omitted as appropriate.

### Embodiment 1

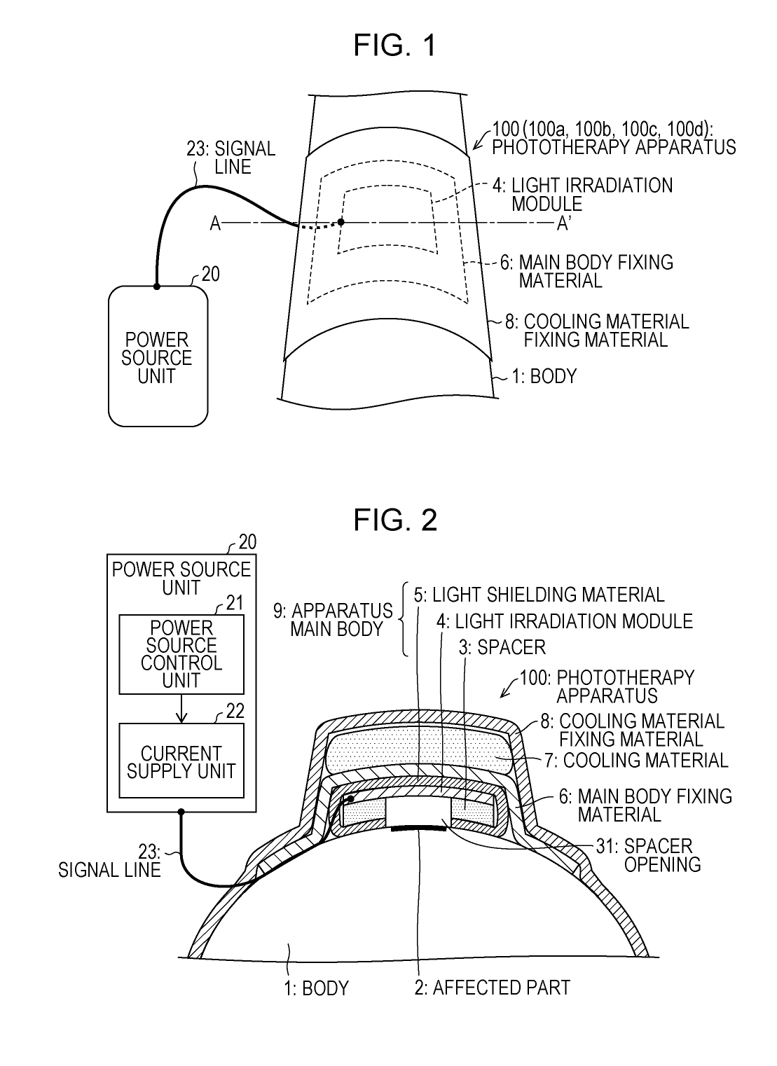

Embodiment 1 of the invention will be described with reference to FIGS. 1 to 6. Note that, the embodiment of the invention will be described by exemplifying a case where light irradiation therapy with a light irradiation module is performed for skin disease having a relatively small area without contacting an affected part. FIG. 1 is a bird's eye view illustrating a state where a phototherapy apparatus 100 according to Embodiment 1 is mounted on a body. More specifically, the bird's eye view illustrates a state when the phototherapy apparatus 100 is mounted on the body to treat an affected part 2 (part to be treated) developed in the body 1, such as a hand, a foot, or a face. FIG. 2 is a schematic sectional view illustrating the state where the phototherapy apparatus 100 is mounted on the body. More specifically, FIG. 2 is obtained by adding a functional block of a power source unit 20 to a sectional view taken along a line A-A′ in FIG. 1.

### (Configuration of Phototherapy Apparatus)

The phototherapy apparatus 100 according to the present embodiment includes a spacer 3, a light irradiation module 4, a light shielding material 5 (light shielding unit), a main body fixing material 6, a cooling material 7, a cooling material fixing material 8, a power source unit 20, and a signal line 23 as illustrated in FIGS. 1 and 2.

The light irradiation module 4 radiates light for treatment when current is supplied from the power source unit 20. The light irradiation module 4 is connected to the power source unit 20 via the signal line 23 and radiates light when predetermined current is supplied from the power source unit 20.

The spacer 3 is disposed on a light output side of the light irradiation module 4, that is, a side facing the affected part 2, and contacts a surface of the body 1 having the affected part 2 to keep a distance between the light irradiation module 4 and the affected part 2 constant. In the spacer 3, a spacer opening (opening) 31 that avoids contact of the affected part 2 and the spacer 3 is formed. In other words, the spacer opening 31 into which the affected part 2 enters in a state where the spacer 3 contacts the surface of the body 1 is formed. The spacer opening 31 is formed near a center of a surface of the spacer 3, which is on the affected part 2 side. Note that, though the spacer 3 indirectly contacts the surface of the body 1 via the light shielding material 5 in FIG. 2, such indirect contact is also included in the contact described above. A function of the spacer 3 is to keep the distance between the light irradiation module 4 and the affected part 2 constant and a distance between the light irradiation module 4 and the surface of the body 1 around the affected part 2 constant. This prevents heat generated from the light irradiation module 4 during light emission from being transferred to the affected part 2 or the surface of the body around the affected part 2, and also makes it possible to achieve uniform distribution of light irradiation intensity. Note that, a lower surface of the spacer 3 may directly or indirectly contact the surface of the body 1.

Since the spacer opening 31 is provided, the affected part 2 does not directly contact the spacer 3 or the light irradiation module 4. When the affected part 2 and the spacer opening 31 are arranged so as to face each other, the affected part 2 faces the light irradiation module 4 with an air layer therebetween and is able to receive light irradiation from the light irradiation module 4.

The light shielding material 5 shields light from the light irradiation module 4 and covers the spacer 3 and the light irradiation module 4, which are stacked, from a back surface side of the light irradiation module 4. By covering the spacer 3 and the light irradiation module 4 with the light shielding material 5, light output from the light irradiation module 4 is able to be prevented from leaking to outside from a part other than a surface on which the spacer opening 31 is formed and which faces the body 1. Since the light emitted from the light irradiation module 4 during treatment is powerful, a patient or a medical practitioner is protected from the light by wearing special protective equipment, however, the light becomes difficult to be leaked to outside, so that the patient or the medical practitioner does not need to wear such special protective equipment.

The light shielding material 5 covers the surface of the spacer 3, which is on the affected part 2 side, other than the spacer opening 31 or other than the spacer opening 31 and an edge thereof. This makes it possible to reduce unnecessary light irradiation to a normal part of skin around the affected part 2 when light is radiated from the light irradiation module 4, and reduce a side effect on the normal part of the skin. Note that, the light shielding material 5 is not essential to perform phototherapy without directly contacting the affected part 2. This is because, for example, also by imparting a light shielding property to the main body fixing material 6 and/or the cooling material fixing material 8 without using the light shielding material 5, leakage of therapeutic light to outside is able to be prevented. Also in prevention of a side effect on a normal part around the affected part 2, by arranging a light shielding material, which is different from the light shielding material 5, on the surface of the body 1, leakage of therapeutic light to outside is able to be prevented.

The main body fixing material 6 fixes, to the body 1, the apparatus main body 9 in which the light irradiation module 4 and the spacer 3 that are stacked are covered with the light shielding material 5.

The cooling material 7 cools the light irradiation module 4. The cooling material 7 is disposed so as to cover a back surface of the apparatus main body 9 that is fixed to the body 1 by the main body fixing material 6 and the cooling material 7 that is disposed in this manner is fixed to the body 1 by the cooling material fixing material 8.

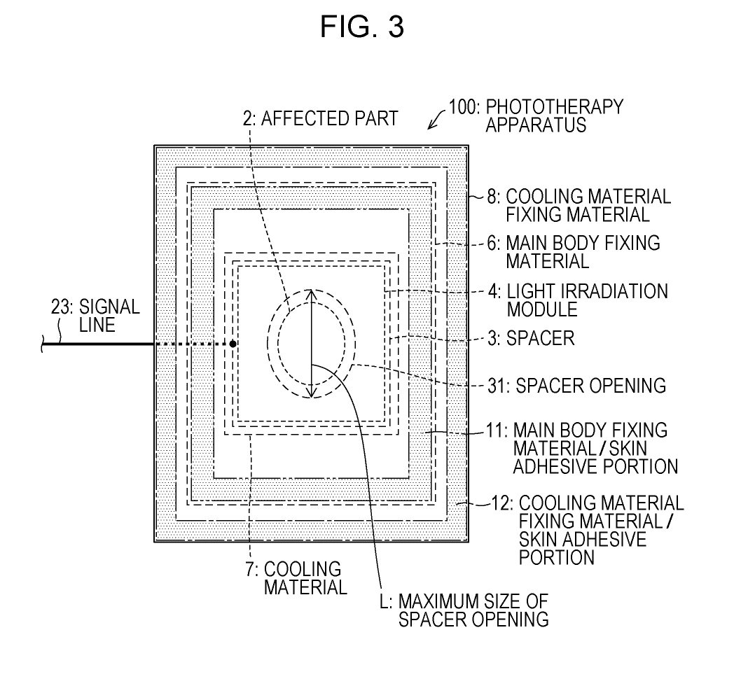

In FIG. 3 described below, as each of the main body fixing material 6 and the cooling material fixing material 8, a skin adhesive one is exemplified. However, the phototherapy apparatus 100 may be fixed to skin in such a manner that the main body fixing material 6 adheres to the cooling material fixing material 8 and the cooling material fixing material 8, to which the main body fixing material 6 adheres, adheres to the skin. This makes it possible to minimize an area of a part adhering to the skin. In this case, since the main body fixing material 6 adheres to the cooling material fixing material 8 in advance, a work of causing the main body fixing material 6 to adhere to the skin does not need to be performed. Thus, it is possible to reduce burden when the phototherapy apparatus 100 is mounted on the body 1. It is also possible, with the main body fixing material 6 omitted, to fix the apparatus main body 9 and the cooling material 7 to the body. 1 by the cooling material fixing material 8 in a state where the cooling material 7 is directly placed on the apparatus main body 9. In a case where heat generation from the light irradiation module 4 is small and the cooling material 7 is not necessary, the cooling material fixing material 8 is able to be omitted.

The power source unit 20 supplies current to the light irradiation module 4. The power source unit 20 includes a power source control unit 21 and a current supply unit 22. The power source control unit 21 controls the current supplied to the light irradiation module 4 and the current supply unit 22 supplies the current to the light irradiation module 4. The power source unit 20 and the light irradiation module 4 are connected by the signal line 23.

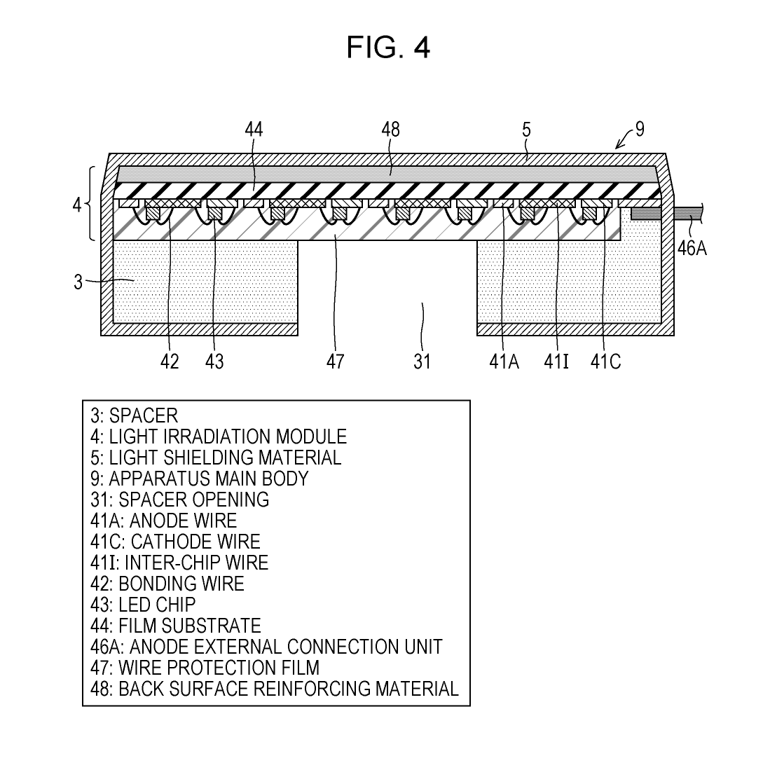

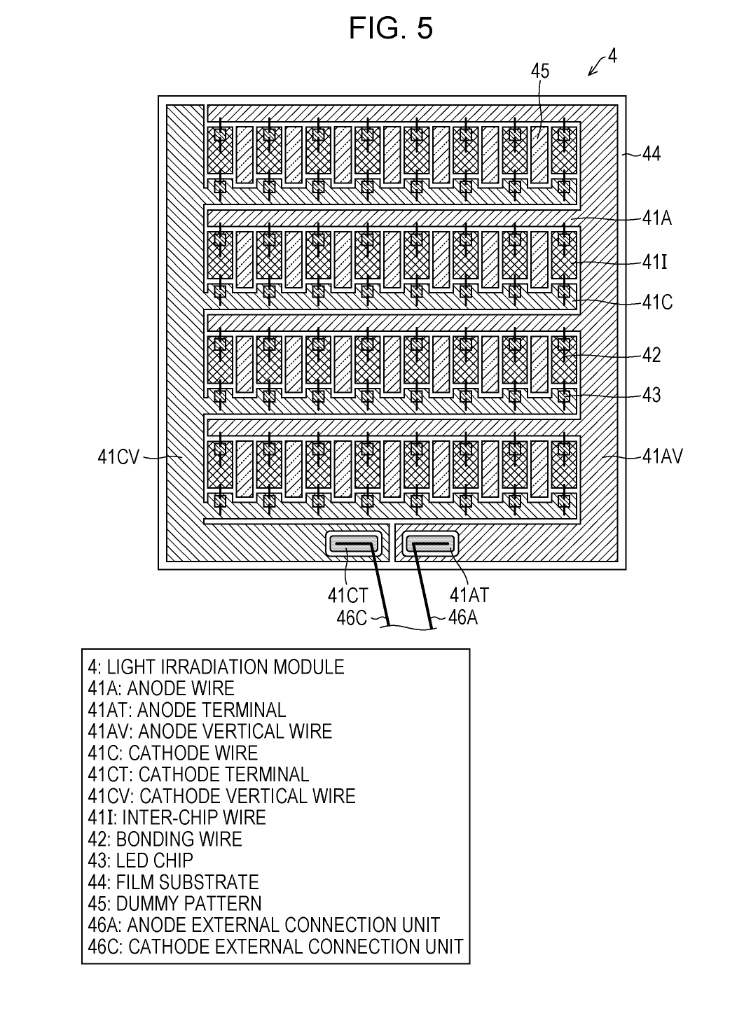

The phototherapy apparatus 100 will be described in detail below with reference to FIGS. 3 to 6. FIG. 3 is a plan view illustrating the state where the phototherapy apparatus 100 is mounted on the body. FIG. 4 is a sectional view of the apparatus main body 9 of the phototherapy apparatus 100. FIG. 5 is a plan view of the light irradiation module 4. In FIG. 5, illustration of a wire protection film 47 described below is omitted.

The light irradiation module 4 is assumed to include, for example, a film substrate in which an LED (Light Emitting Diode) serving as a light emitting element that emits a predetermined wavelength is mounted on a resin surface having flexibility so as to enable lighting. Moreover, in combination with light for treatment, specific medicine has been normally applied to the affected part 2 or taken in advance in many cases. It is assumed that an appropriate distance is kept between the LED and the affected part 2 to uniformly perform light irradiation for the affected part 2. However, medicine, a light wavelength which is used for treatment, details of the film substrate, or the like does not affect the present embodiment and is thus not described in detail here.

### (Details of Configuration of Light Irradiation Module)

The light irradiation module 4 includes a film substrate 44 as illustrated in FIGS. 4 and 5. In the film substrate 44, on a light emitting surface (light output surface) that is a surface on the spacer 3 side, anode wires 41A, cathode wires 41C, inter-chip wires 41I, an anode vertical wire 41AV, a cathode vertical wire 41CV, an anode terminal 41AT, and a cathode terminal 41CT are provided, and LLD chips 43 serving as light sources are arranged thereon in an array.

Specifically, as illustrated in FIG. 5, the film substrate 44 has a square shape and the anode terminal 41AT and the cathode terminal 41CT are provided side by side along one side of the film substrate 44. However, the film substrate 44 may have, for example, a rectangular shape, a circular shape, an elliptical shape, a rectangular shape with rounded corners, or the like.

From the anode terminal 41AT, an anode wire 41A extends along the one side toward a direction opposite to the cathode terminal 41CT, and is connected to the anode vertical wire 41AV that extends along a side adjacent to the one side. Further, from the anode vertical wire 41AV, the anode wires 41A are arranged at regular intervals in a direction parallel to the one side, and the LED chips 43 are connected to the wires at regular intervals.

Similarly to the anode wire 41A, from the cathode terminal 41CT, a cathode wire 410 also extends along the one side toward a direction opposite to the anode terminal 41AT and is connected to the cathode vertical wire 41CV that extends along a side adjacent to the one side. Further, from the cathode vertical wire 41CV, the cathode wires 41C are arranged at regular intervals in a direction parallel to the one side, and the LED chips 43 are connected to, the wires at regular intervals.

An LED chip 43 connected to the anode wire 41A and an LED chip 43 connected to the cathode wire 41C are arranged facing each other and an inter-chip wire 41I is wired therebetween. A dummy pattern 45 is arranged between adjacent inter-chip wires 41I.

The LED chips 43 are arranged on wires and connected to the respective wires by bonding wires 42. Various light emitting elements such as ultraviolet, blue-violet, blue, green, red, and infrared light emitting elements are selectable as the LED chips 43 in accordance with a therapeutic purpose note that, though an example of wire bonding connection is described in FIG. 5, the LED chips 43 and the respective wires may be connected by flip chip connection. In a case where each of the LED chips 43 has a vertical electrode structure, one terminal (normally, negative electrode) is connected to a wire by conductive paste and the other terminal is connected by wire bonding. The light irradiation module 4 that uses the LED chips 43 as light emitting elements have excellent flexibility.

Note that, as a light emitting element, an LED device in which LED chips are stored in a package may be used in place of the LED chips 43 themselves. The light irradiation module 4 using the LED device is able to have increased mechanical strength.

The anode wires 41A and the cathode wires 41C are connected to an anode external connection unit 46A and a cathode external connection unit 46C at the anode terminal 41AT and the cathode terminal 41CT, respectively, and connected to the power source unit 20 via the signal line 23. For example, a lead wire is able to be used as the anode external connection unit 46A and the cathode external connection unit 46C.

The wire protection film 47 covers and protects the respective wires, the LED chips 43, and connected parts thereof. The wire protection film 47 is preferably made from a resin material, such as silicone resin, which is transparent or has a light-transmitting property and an excellent moisture proofing property.

The film substrate 44 may be made from an insulating material having flexibility. For example, it is possible that a film substrate is a polyimide film and wires are made by etching copper foil and silver electroplating. A material of the respective wires preferably has low resistance and high reflectance of a front surface. The film substrate 44 may be a polyimide film having a thickness of about 25 μm.

In a case where the film substrate 44 is constituted by a material having a high light absorbing property like a polyimide film, a configuration in which a wire material having high reflectance covers an area of a front surface of the film substrate 44 as wide as possible, for example, by arranging dummy patterns 45 as illustrated in FIG. 5 is preferable. On the other hand, in a case where the film substrate 44 is constituted by a material having high reflectance like a resin material in which white pigment is kneaded, the dummy patterns 45 as illustrated in FIG. 5 are not necessarily required to be provided, and the respective wires are able to have a reduced width in a range where a voltage drop does not matter. The configuration may be also such that a surface of the wire material is subjected to gold plating. The gold plating achieves high reflectance in a range from red light to infrared light, and is thus suitable for a phototherapy apparatus using such light. Since the wire material subjected to the gold plating has longterm stability and excellent durability and thus enables loosening of a saving condition such as humidity. As a result, as for the film substrate 44, it is preferable that at least the front surface of the film substrate 44 has high total reflectance with respect to therapeutic light in order to efficiently irradiate the affected part 2 with the therapeutic light.

The light irradiation module 4 desirably has certain strength to suppress deformation of the light irradiation module 4 at the spacer opening 31. The light irradiation module 4 needs strength for preventing local sinking of the light irradiation module 4 at the spacer opening. 31 while having flexibility which allows deformation along a surface of a body. When the light irradiation module 4, the light shielding material 5, the main body fixing material 6, the cooling material 7, and the cooling material fixing material 8 sink toward the spacer opening 31 and are thus deformed, light irradiation intensity may be excessively increased in a center of the spacer opening 31 compared with that in a peripheral part of the spacer opening 31. When such deformation becomes great, a distance between the LED chips 43 of the light irradiation module 4 and the affected part 2 becomes short and the affected part 2 facing the LED chips 43 is to receive locally strong light irradiation. To prevent this, the strength of the light irradiation module 4 needs to be increased. As a method of increasing the strength of the light irradiation module 4, a film thickness of the film substrate 44 is increased. The film thickness of the film substrate 44 is preferably, for example, 20 μm or more, and more preferably 30 μm or more.

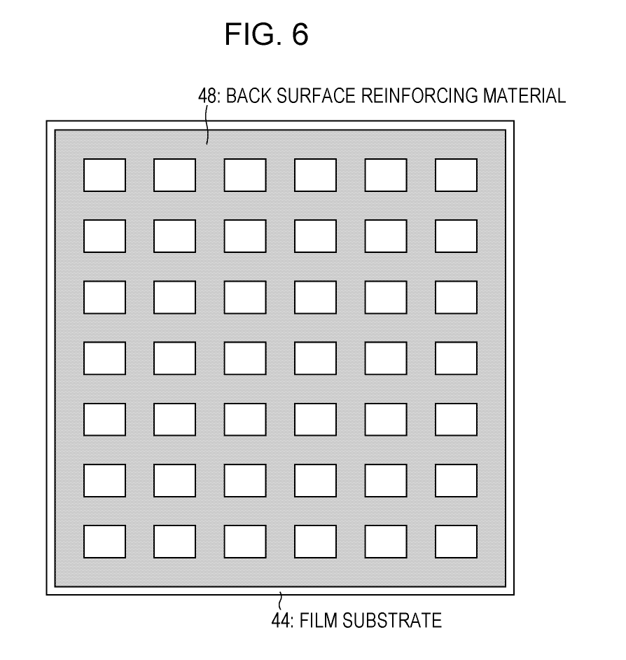

As another method of increasing the strength of the light irradiation module 4, a back surface reinforcing material 48 is provided on a back surface (surface opposite to the spacer 3 side) of the film substrate 44 as illustrated in FIG. 6. As a material thereof, metal foil, such as copper foil or stainless steel foil, having a thickness of about several μm to 25 μm is preferable. The back surface reinforcing material 48 does not need to have a mesh shape as illustrated in FIG. 6 and may have a fish bone shape or a simple rectangular shape to achieve both flexibility and strength. Thereby, the strength of the light irradiation module 4 is able to be increased, thus making it possible to prevent local sinking of the light irradiation module 4 at the spacer opening 31 and prevent an excessive increase in the light irradiation intensity in the center of the spacer opening 31 compared with that in the peripheral part of the spacer 3.

The light irradiation module 4 preferably includes a temperature sensor that enables an interlocking function of stopping current, which flows through the light irradiation module 4, to stop light emission when temperature of the light irradiation module 4 rises beyond an upper limit thereof. In addition, the light irradiation module 4 preferably includes a safety device, such as a current breaker, which breaks current flowing through the light irradiation module 4 to stop light emission, for example, in an abnormal case where the light irradiation module 4 is dropped out from the affected part 2. The light irradiation module 4 may also include a light intensity sensor. The light intensity sensor monitors light intensity of light emitted by the light irradiation module 4, and when an irradiation amount of the light of the light irradiation module 4 exceeds a predetermined amount, stops light irradiation of the light irradiation module 4.

### (Spacer)

The spacer 3 is disposed on the light output side of the light irradiation module 4, and keeps a distance between the surface of the body 1 and the light irradiation module 4 constant. In the configuration of the present embodiment, the affected part 2 is irradiated with light through the spacer opening 31, so that the spacer 3 is not necessarily required to have a light-transmitting property. However, the spacer 3 preferably has a light-transmitting property to effectively utilize light radiated from the LED chips 43 arranged on a part other than the part corresponding to the spacer opening 31. It is also preferable that a surface of the spacer 3 and a surface of the light irradiation module 4 that are in contact closely adhere to each other. When there is an air layer between the spacer 3 and the light irradiation module 4, reflection of light occurs at an interface between the light irradiation module 4 and the spacer 3, thus causing a loss of light.

The spacer 3 has a function of uniformizing the light emitted from the light irradiation module 4. The spacer 3 also has a function as a heat insulator that does not allow heat generated by the light irradiation module 4 to be directly transferred to the skin.

In the spacer opening 31, the affected part 2 and the light irradiation module 4 are separated from each other by a thickness of the spacer 3, so that the spacer 3 achieves the function of not allowing the heat generated by the light irradiation module 4 to be directly transferred to the affected part 2. Since the spacer 3 prevents the heat from the light irradiation module 4 from being directly transferred to the affected part 2 or the skin around the affected part 2, a temperature rise of the light irradiation module 4 is able to be permitted to a certain degree, so that a large-sized cooling device is not required. Furthermore, in the phototherapy apparatus 100, treatment is finished in a relatively short time, so that a cooling water circulation device or the like does not need to be installed and a patient is not restrained during the treatment.

The spacer 3 preferably has a plate shape that has a constant thickness and is constituted by a flexible and transparent material. For example, the spacer 3 is preferably made from a resin material such as styrene elastomer, urethane rubber, or silicone rubber. Since the LED chips 43 are arranged in an array in the light irradiation module 4, the distance between the affected part 2 and the spacer 3 needs to be kept constant so that the affected part 2 receives uniform light irradiation. The spacer 3 also has a function as a heat insulator for preventing the affected part 2 from feeling cold when the cooling material 7 is used.

As the thickness of the spacer 3 increases, more uniformized light irradiation and a higher heat insulating effect are achieved. When a curvature of a typical human body is considered, a suitable thickness of the spacer 3 is about 2 mm to 10 mm, and a most suitable thickness thereof is about 3 mm to 7 mm. The spacer 3 of a plate shape is difficult to be fitted around a finger, a tip of a nose, or the like and preferably has another form.

An external shape of the light irradiation module 4 is assumed to have almost the same size as that of an external shape of the spacer 3. In this case, when a width of a part where an outer edge (edge of a square shape) of the spacer 3 and an edge (edge of a circular shape) of the spacer opening 31 are closest is smaller than L/2 which is a half of a maximum size L of the spacer opening, the light irradiation module 4 is easily deformed at a part of the spacer opening 31. To prevent this, with respect to the maximum size L of the spacer opening, the width of the part where the edges are closest is preferably at least L/2. Thus, the maximum size L of the spacer opening is preferably equal to or less than a half of the size of the spacer in the same direction. Here, the size of the external shape of the spacer 31 and the size of the external shape of the light irradiation module 4 are preferably 20 mm to 100 mm, and more preferably 30 mm to 60 mm.

The shape of the spacer 3 may not be a square shape and may be a shape, for example., such as a rectangular shape, a circular shape, an elliptical shape, or a rectangular shape with rounded corners. The shape of the spacer 3 is selectable in accordance with a shape of the affected part 2. Since the spacer 3 and the light irradiation module 4 are stacked with each other, the spacer 3 and the light irradiation module 4 preferably have almost equal shapes. In particular, since the spacer 3 and the light irradiation module 4 are covered with the light shielding material 5, it is preferable that the light irradiation module 4 does not protrude from the spacer 3 when the spacer sand the light irradiation module 4 are stacked. That is, the external shape of the light irradiation module 4 is preferably smaller than the external shape of the spacer 3.

Note that, a sectional shape of the spacer opening 31 exemplified in FIG. 4 is a rectangular shape perpendicular to the front surface of the light irradiation module 4, but is not necessarily required to be the rectangular shape and may be a trapezoid shape or an inverted trapezoid shape, or may be a curved surface shape such as a dome shape or an inverted pan-bottom shape.

(Light Shielding Material)

The light shielding material 5 prevents light from the light irradiation module 4 from being radiated to an unnecessary part or leaking to outside. The light shielding material 5 is provided so as to cover the spacer 3 and the light irradiation module 4 as illustrated in FIG. 4. Specifically, the light shielding material 5 covers a part around the spacer 3 and the light irradiation module 4 other than the spacer opening 31 or other than the spacer opening 31 and the edge thereof, in a state where a patient wears the phototherapy apparatus 100. Thereby, the light shielding material 5 shields leakage of the light, with which the affected part 2 is irradiated by the light irradiation module 4, to a part other than the affected part 2. In other words, the light shielding material 5 is arranged between the light irradiation module 4 and the main body fixing material 6 and completely covers the light irradiation module 4 other than the spacer opening 31 (or the spacer opening 31 and the edge thereof), thus making it possible to reduce leakage of the light to outside to an almost negligible degree. At this time, the light shielding material 5 does not cover the spacer opening 31 and thus does not prevent light irradiation to the affected part 2.

The reflectance of the light shielding material 5 is desirably high at a part covering the spacer 3 so as to enable reflection of the light from the light irradiation module 4. In other words, the light shielding material 5 is constituted by a material that reflects light. Thereby, with the reflection by the light shielding material 5, the light is able to reach the affected part 2, though only a little, thus making it possible to increase irradiation efficiency to the affected part 2. The light shielding material 5 is only required to cover at least an outer edge of a bottom surface of the spacer 3 as long as leakage of the light to outside is able to be prevented. However, to prevent light irradiation to a normal part of the skin, the entire bottom surface of the spacer 3 is preferably covered. A case where the film substrate 44 does not transmit light or the film substrate 44 transmits light but the back surface reinforcing material 48 does not transmit light is considered. In such a case, since the film substrate 44 or the back surface reinforcing material 48 functions as the light shielding unit, the light shielding material 5 does not need to cover the entire back surface (surface opposite to the spacer 3 side) of the light irradiation module 4.

The light shielding material 5 is most preferably made from aluminum foil having a thickness of 25 μm to 200 μm, but may be made from a composite material in which a resin film is stacked on thin aluminum foil (having a thickness of about 25 μm). Additionally, the light shielding material 5 may be made from a composite material in which a silver thin film or a gold thin film is formed on copper foil or a resin film, or metal foil in which a gold thin film provided on a surface of aluminum foil. Since a metal material having high reflectance varies depending on a type of light, a reflection material to be used for the light shielding material 5 needs to be changed in accordance with a wavelength range of light.

The light shielding material 5 is not necessarily, required to be constituted by a single member. For example, the spacer 3, an end of the light irradiation module 4, and the outer edge of the back surface (surface opposite to the spacer 3 side) of the light irradiation module 4 may be constituted by aluminum foil or the like having high reflectance as described above. A resin film may be arranged on the back surface (surface opposite to the spacer 3 side) of the light irradiation module 4. Further, as a member preventing leakage of light, a resin material or the like having a light absorbing property is preferably arranged at a part of the light shielding material 5, through which the signal line 23 passes.

### (Main Body Fixing Material)

The main body fixing material 6 fixes the apparatus main body 9, in which the spacer 3, the light irradiation module 4, and the light shielding material 5 are integrated, at a position where the affected part 2 is covered. Specifically, as illustrated in FIGS. 1 and 2, the main body fixing material 6 covers the entire apparatus main body 9 in a planar manner to fix the apparatus main body 9 to a part of the body 1, which has the affected part 2.

The main body fixing material 6 is preferably configured to include a main body fixing material/skin adhesive portion 11 that fixes the main body fixing material 6 to the body 1, for example, as illustrated in FIG. 3. This makes it possible to bond and fix the main body fixing material 6 to the skin around the affected part 2 and also possible to firmly fix the apparatus main body 9 to the body 1.

In this case, a plurality of surgical tape strips may be used as the main body fixing material 6 to fix the apparatus main body 9 to the skin. The main body fixing material 6 as thin as possible is preferably used so that the cooling material 7 is able to cool the light irradiation module 4.

The main body fixing material 6 may be a sheet or tape that has flexibility and is constituted by cloth, leather, nonwoven fabric, a resin film, rubber, artificial leather, synthetic leather, metal foil, paper, or the like, or a composite material thereof. As the main body fixing material 6, one having an elastic property is used. This makes it possible to perform pressurization against the body 1 so that the light irradiation module 4 closely adheres to the skin.

The main body fixing material 6 preferably has a size completely covering at least the light shielding material 5 (that is, apparatus main body 9). However, since a purpose of the main body fixing material 6 is to fix the apparatus main body 9 to the body 1, the apparatus main body 9 may be partially exposed to outside when the phototherapy apparatus 100 is mounted on the body 1. For example, surgical tape, which is made in a crisscross pattern, may be attached to the apparatus main body 9 and the body 1 to fix the apparatus main body 9 to the body 1. The main body fixing material 6 preferably has a flat sheet shape before being mounted on the affected part 2.

When the main body fixing material 6 is mounted on the body 1, first, the light shielding material 5 that covers a periphery of the spacer 3 and the light irradiation module 4 is pressed against the body 1 so that positions of the spacer opening. 31 and the affected part 2 match. That is, the apparatus main body 9 covered with the light shielding material 5 is pressed against the body 1. The main body fixing material 6 covers the light shielding material 5, that is, the apparatus main body 9 covered with the light shielding material 5. At this time, in a state where both ends of the main body fixing material 6 are pulled, and further, a predetermined pressure is applied to the light shielding material 5, that is, the apparatus main body 9 covered with the light shielding material 5 toward the body 1, the main body fixing material 6 is fixed to the body 1 by the main body fixing material/skin adhesive portion 11. Thereby, the main body fixing material 6 is able to fix a position of the light shielding material 5 that covers the periphery of the spacer 3 and the light irradiation module 4, that is, the apparatus main body 9 covered with the light shielding material 5, with respect to the body 1.

### (Cooling Material)

The cooling material 7 cools the light irradiation module 4. In a case where the light irradiation module 4 generates great heat and may have temperature of 42° C. or more within a predetermined treatment time, cooling is required. When the cooling material 7 that is cooled (for example, from about 0° C. to 15° C.) in advance is arranged on the back surface of the light irradiation module 4 for cooling, a temperature rise of the light irradiation module 4 is able to be suppressed. In the phototherapy apparatus 100, treatment is performed in a relatively short time, so that a large-sized cooling device that is required in long-time treatment and circulates water becomes unnecessary. Since the main body fixing material 6, the light shielding material 5, the light irradiation module 4, and the spacer 3 are between the cooling material 7 and the body 1, the cooling material 7 does not directly contact the body 1 and a patient does not feel very cold.

As the cooling material 7, a gel material that is generally used and made from sodium polyacrylate and water is preferably used in terms of costs. A bag in which water is simply enclosed is also usable as the cooling material 7. For example, water that is cooled to 10° C. or less in a normal refrigerator or the like is also usable as the cooling material 7. Water at 0° C. or less is also usable as the cooling material 7.

In addition, an instantaneous cooling material that is made from water and urea/ammonium nitrate or the like is also usable as the cooling material 7. In the instantaneous cooling material, water and urea/ammonium nitrate are enclosed in different rooms, and when the resultant is hit from outside to break a wall between both of them, cooling is achieved by endothermic reaction caused by mixture, so that temperature is able to be reduced to 15° C. or less in about one minute.

### (Cooling Material Fixing Material)

The cooling material fixing material 8 fixes the cooling material 7 onto the main body fixing material 6. A member similar to that of the main body fixing material 6 may be used for the cooling material fixing material 8. While, for the main body fixing material 6, it is necessary to consider enabling the cooling material 7 to cool the light irradiation module 4, such consideration is not required for the cooling material fixing material 8, so that the cooling material fixing material 8 may be a stretchable bandage, stretchable mesh, or the like. Note that, the cooling material fixing material 8 completely covers the main body fixing material 6 in FIG. 3. However, since a purpose of the cooling material fixing material 8 is to fix the cooling material 7 to the apparatus main body 9, the cooling material 7 and/or the main body fixing material 6 may be partially exposed from the cooling material fixing material 8 when the phototherapy apparatus 100 is mounted on the body 1.

In the present embodiment, the cooling material fixing material 8 has a cooling material fixing material/skin adhesive portion 12 as illustrated in FIG. 3 and adheres to the body 1 by using the cooling material fixing material/skin adhesive portion 12.

### (Power Source Unit and Signal Line)

The phototherapy apparatus 100 includes the power source unit 20 that supplies current to the light irradiation module 4. The power source unit 20 includes the power source control unit 21 and the current supply unit 22. The power source control unit 21 instructs the current supply unit 22 to supply or stop the current to the light irradiation module 4. The current supply unit 22 is a current source that supplies the current to the light irradiation module 4.

The current supply unit 22 supplies predetermined current to the light irradiation module 4 and thereby causes the light irradiation module 4 to radiate light for a predetermined time period. The power source control unit 2 controls a current amount of the current supplied by the current supply unit 22, ON/OFF of the current, and an irradiation time of the light irradiation module 4. For example, in a case where current of 400 mA is supplied to the light irradiation module 4 illustrated in FIG. 5, in which 64 blue-violet light emitting diodes (peak wavelength 405 nm) are arrayed in 8 rows and 8 columns with a pitch of 5 mm, driving voltage of about 7 V is required. The current of 12.5 mA flows through each of the LED chips 43 and light irradiation intensity of 37.5 mW/cm² is obtained near a center thereof. In a case where a dose amount of energy required for treatment is 25 J/cm², irradiation is able to be completed in about eleven minutes. The irradiation time is assumed to be one hour or less at the longest, and the treatment is normally completed in about several minutes to twenty minutes. Thus, power is not necessarily required to be obtained from an AC power source and a dry battery or a storage battery may be used as the power source.

The power source unit 20 has a function of setting a required current amount and a required time in accordance with target treatment and a function of, in actual treatment, starting irradiation and stopping the irradiation after a predetermined time has lapsed. The power source unit 20 preferably has an interlocking function of stopping current to the light irradiation module in an abnormal case where temperature of the light irradiation module 4 rises, the light irradiation module 4 is dropped out, or the like.

Here, Examples 1 and 2 of the phototherapy apparatus 100 are provided and a result of checking presence or absence of deformation or the like will be described. Example 1 and Example 2 are different in a thickness of the film substrate 44 in the light irradiation module 4, a thickness of copper foil of respective wire patterns formed on the front surface of the film substrate 44, and presence or absence of the back surface reinforcing material 48.

In the phototherapy apparatus 100 of Example 1, the thickness of the film substrate 44 is 25 μm, the thickness of the copper foil constituting the wire patterns is 20 μm, and the back surface reinforcing material 48 is provided. On the other hand, in the phototherapy apparatus 100 of Example 2, the thickness of the film substrate 44 is 12.5 μm, the thickness of the copper foil constituting the wire patterns is 10 μm, and the back surface reinforcing material 48 is not provided.

In the phototherapy apparatus 100 of Example 2, compared with the phototherapy apparatus 100 of Example 1, the thickness of the film substrate 44 and the thickness of the copper foil are small and the back surface reinforcing material 48 is not provided, so that high flexibility is achieved. However, when the main body fixing material 6, the cooling material 7, and the cooling material fixing material 8 are installed, deformation is found at a part of the spacer opening 31 depending on a size of the spacer opening 31. Specifically, in a case where a diameter of the spacer opening 31 is 10 mm, there is no problem, but in a case of 20 mm, downward looseness by about 1 mm is found at a center of the spacer opening 31. In a case where the diameter of the spacer opening 31 is 30 mm, downward looseness by about 2.5 mm is found.

As a result, it is found that, in a case where the diameter of the spacer opening 31 is long, it is preferable that the thickness of the film substrate 44 and the thickness of the copper foil constituting the wire patterns are increased and the back surface reinforcing material 48 is provided.

### Embodiment 2

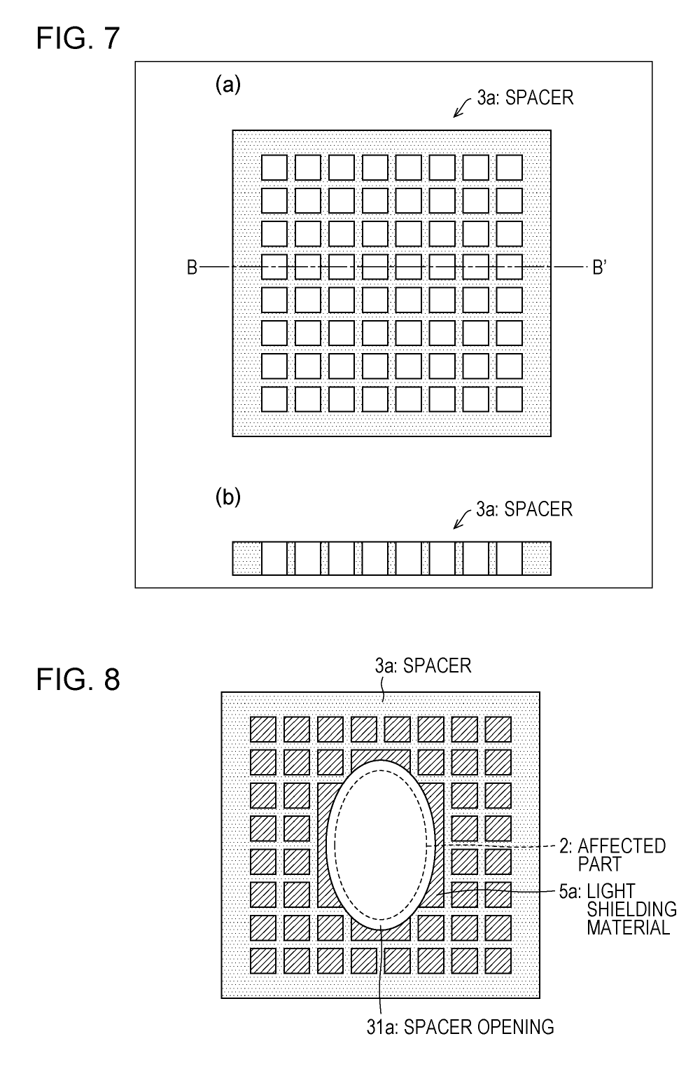

Embodiment 2 of the invention will be described as follows with reference to FIGS. 7 and 8. Note that, for convenience of description, a member having the same function as that of the member described in the foregoing embodiment will be given the same reference sign and description thereof will be omitted. FIG. 7(a) is a plan view of a spacer 3a of a phototherapy apparatus 100a according to Embodiment 2 and FIG. 7(b) is a sectional view of the spacer 3a illustrated in FIG. 7(a).

The phototherapy apparatus 100a according to the present embodiment is different from the phototherapy apparatus 100 in that the spacer 3 is different from the spacer 3a and the light shielding material 5 is changed to a light shielding material 5a with the change to the spacer 3a, and is the same as the phototherapy apparatus 100 in others (refer to FIG. 1).

The spacer 3a is formed in a lattice pattern as illustrated in FIGS. 7(a) and 7(b). By cutting a partition part (lattice part) of the lattice pattern in the spacer 3a as illustrated in FIG. 8, the spacer opening 31a is formed in accordance with a size and a shape of the affected part 2. When the spacer 3a is formed in the lattice pattern, the spacer opening 31a is able to be easily formed.

That is, the partition part of the lattice pattern is narrow, and is thus easily cut by a tool such as scissors or nippers. Therefore, a size of the spacer opening. 31a is able to be matched with the size of the affected part 2. Thus, the size of the spacer opening 31a is able to be reduced to a necessary minimum size. As a result, since the size of the spacer opening 31a is able to be reduced, deformation of the light irradiation module 4 at the spacer opening 31a is able to be suppressed.

Though a bottom surface of the light shielding material 5a may be largely opened in advance, an opening is preferably provided in the light shielding material 5a in accordance with a shape of the spacer opening 31a. That is, it is preferable that a part of the light shielding material 5a is provided on an entire bottom surface of the spacer 3a, and when the spacer opening 31a is provided in the spacer 3a, an opening is also provided in the light shielding material 5a at the same time. As a result, since the size of the spacer opening 31 is able to be minimized, an area of a normal part subjected to light irradiation in skin is able to be reduced to a minimum. Additionally, since the size of the spacer opening 31 is able to be minimized, deformation of the light irradiation module 4 at the spacer opening 31a is able to be suppressed. Note that, a structure of the lattice pattern of the spacer 3a does not need to have one layer as illustrated in FIG. 7(b) and may have multiple layers. Specifically, the structure may be such that a lattice pattern is seen in any surface.

### Embodiment 3

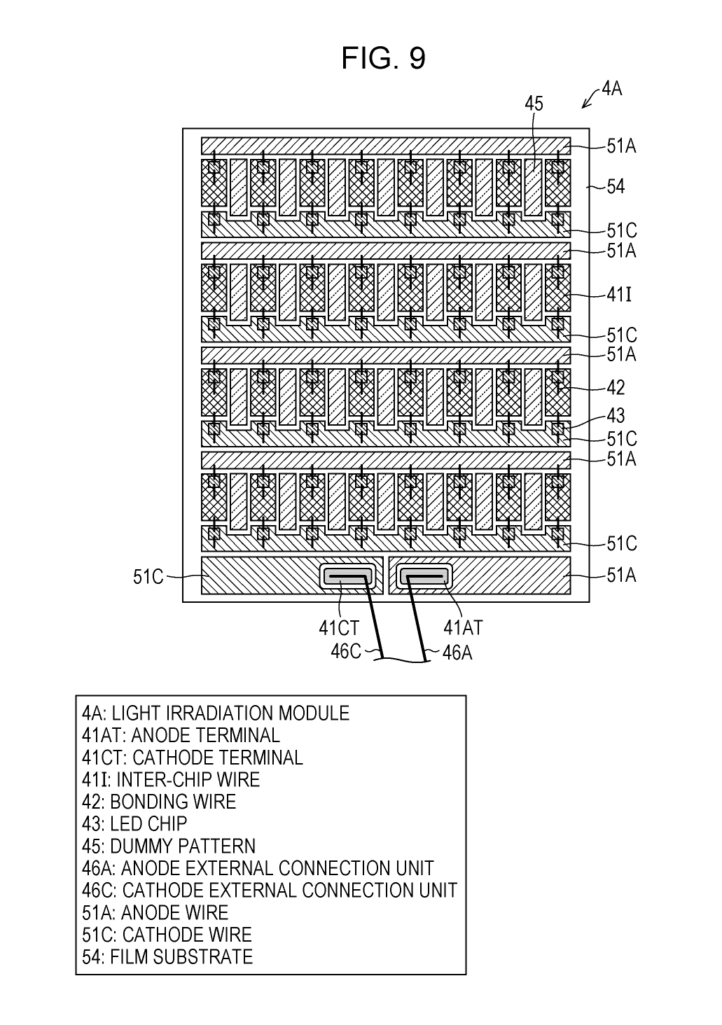

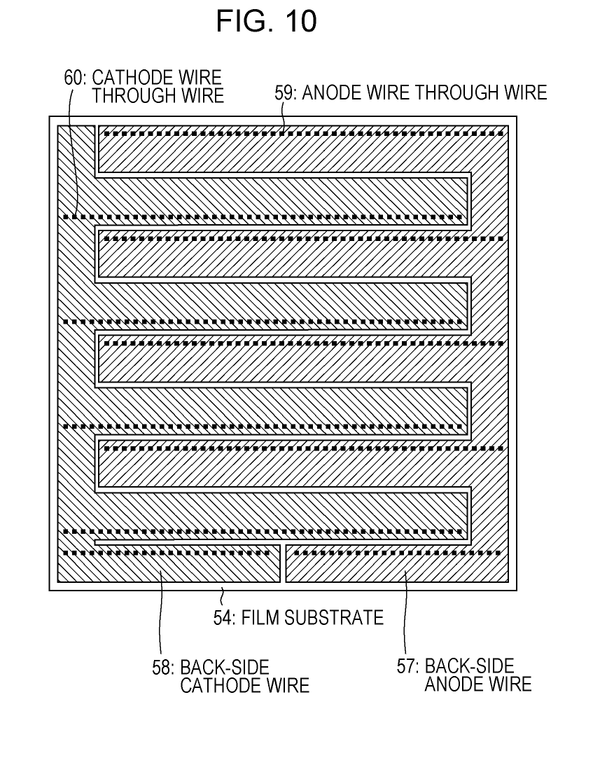

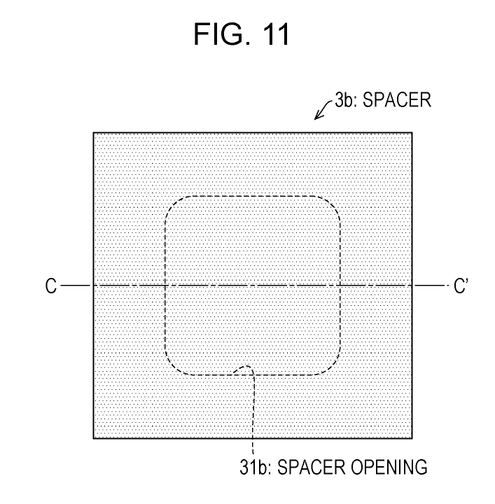

Embodiment 3 of the invention will be described as follows with reference to FIGS. 9 and 10. Note that, for convenience of description, a member having the same function as that of the member described in the foregoing embodiments will be given the same reference sigh and description thereof will be omitted. FIG. 9 is a plan view of a light irradiation module 4A of a phototherapy apparatus 100b according to Embodiment 3. FIG. 10 is a plan view of a back surface of the light irradiation module 4A illustrated in FIG. 9.

The phototherapy apparatus 100b according to the present embodiment is the same as the phototherapy apparatus 100 excluding that there is a change to the light irradiation module 4A (refer to FIG. 1).

As illustrated in FIG. 9, the light irradiation module 4A includes, on a front surface (surface on the spacer 3 side) of a film substrate 54, a plurality of anode wires 51A, a plurality of cathode wires 51C, a plurality of inter-chip wires 41I, the anode terminal 41AT, the cathode terminal 41CT, the dummy patterns 45, a plurality of LED chip 43, a plurality of bonding wires 42, the wire protection film 47 (not illustrated), the anode eternal connection unit 46A, and the cathode external connection unit 46C.

A difference from the light, irradiation module 4 lies in that the light irradiation module 4A includes, on a back surface (surface opposite to the spacer side) of the film substrate 54, a back-side anode wire 57 (second wire), a back-side cathode wire 58 (second wire), anode wire through wires 59, and cathode wire through wires 60 as illustrated in FIG. 10. Each of the anode wire through wires 59 connects a front wire and a back wire of the anode wires and each of the cathode wire through wires 60 connects a front wire and a back wire of the cathode wires. In the light irradiation module 4A, the back-side anode wire 57 and the back-side cathode wire 58 function as a back surface reinforcing material. The anode wire through wires 59 connect the anode wires 51A and the back-side anode wire 57 and the cathode wire through wires 60 connect the cathode wires 51C and the back-side cathode wire 58.

In the light irradiation module 4A, the anode vertical wire 41AV or the cathode vertical wire 41CV does not need to be provided, so that a size of the film substrate 54 is able to be reduced. This makes it possible to easily perform treatment for a part whose area is relatively small, such as a face or an arm, or a part with a large undulation, or treatment for skin of a child.

### Embodiment 4

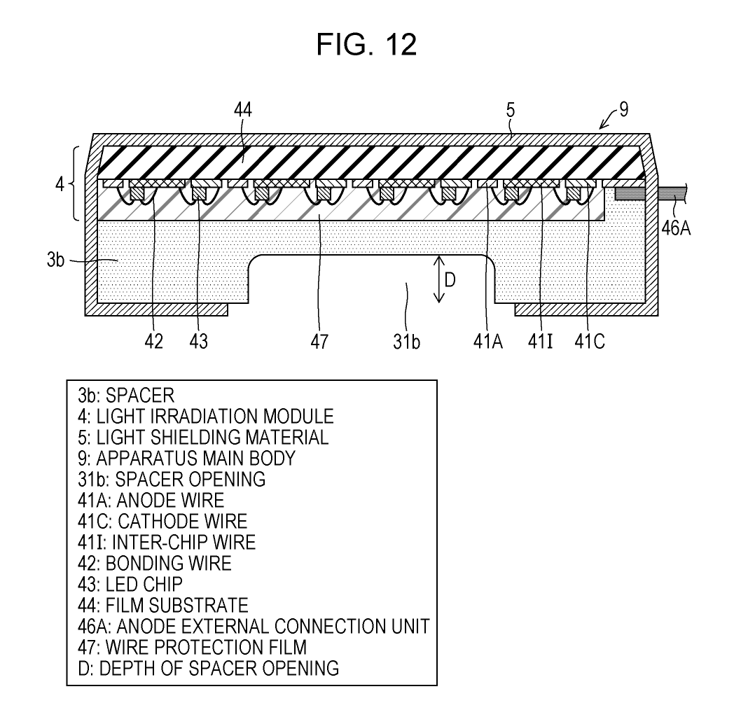

Embodiment 4 of the invention will be described as follows with reference to FIGS. 11 and 12. Note that, for convenience of description, a member having the same function as that of the member described in the foregoing embodiments will be given the same reference sign and description thereof will be omitted. FIG. 11 is a plan view of a spacer 3b of a phototherapy apparatus 100c according to Embodiment 4. FIG. 12 is a sectional view of an apparatus main body of the phototherapy apparatus 100c according to Embodiment 4.

The phototherapy apparatus 100c according to the present embodiment is the same as the phototherapy apparatus 100 excluding that the spacer 3 is changed to the spacer 3b as illustrated in FIG. 11 (refer to FIG. 1). In the spacer 3 of the phototherapy apparatus 100, the spacer opening 31 extends through a front surface (surface on the light irradiation module 4 side) to a back surface (surface on the affected part 2 side) of the spacer 3. On the other hand, in the spacer 3b of the present embodiment, a spacer opening 31b is constituted by a concave part that is formed only on the affected part 2 side and does not reach the front surface (surface on the light irradiation module 4 side) as illustrated in FIGS. 11 and 12.

With such a configuration, the other part of the spacer opening 31b acts as a reinforcing structure, so that deformation of the light irradiation module 4 at the spacer opening 31b is able to be suppressed and prevented without providing the back surface reinforcing material 48. In a case where a thickness of the spacer 3b is 5 mm, a depth D of the spacer opening is suitably about 1 mm to 4 mm (20% to 80% of the thickness of the spacer 3b). In a case where the affected part 2 swells from a surface of skin, the depth D of the spacer opening needs to be increased.

Note that, a planar shape of the spacer opening 31b is a square shape whose corners are rounded as illustrated in FIG. 11, but may be a complete square shape, a circular shape, or an elliptical shape. The shape of the spacer opening 31b may be selected in accordance with the shape of the affected part 2. A sectional shape of the spacer opening 31b illustrated in FIG. 12 is an inverted pan shape (corners of which are curved), but may be a rectangular shape or a shape (inverted Chinese pan shape) whose inclination is gentler. The sectional shape of the spacer opening 31b affects distribution of light irradiation intensity on the affected part 2 facing the spacer opening 31b. Thus, with the arrangement of the LED chips 43 on the light irradiation module 4, the sectional shape of the spacer opening 31b is preferably optimized so as to reduce nonuniformity of the distribution of light irradiation intensity

The light shielding material 5 contacting a surface of the spacer 3b, which is on the affected part 2 side, does not cover up to an edge of the spacer opening 31b as illustrated in FIG. 12, but may cover up to the edge of the spacer opening 31b. In a case where the light shielding material 5 covers also the edge of the spacer opening 31b, by preventing light irradiation to a part other than the spacer opening 31b to reflect more light, light radiated to the spacer opening 31b is able to be increased.

As for a method of manufacturing the spacer 3b, the spacer 3b is able to be manufactured, for example, through mold forming with use of a die. Moreover, without using a die, a simple late part which is to be on the light irradiation module 4 side and a plate part which is to be on a side contacting the body 1 and has a through opening may be separately prepared and the plate parts may be bonded with each other later.

Accordingly, since the spacer opening 31b is provided on the affected part 2 side and does not reach the surface on the light irradiation module 4 side in the phototherapy apparatus 100c, even when the back surface reinforcing material 48 is not provided, local sinking of the light irradiation module 4 is not caused. Thus, the back surface reinforcing material 48 does not need to be provided, so that costs required for providing the back surface reinforcing material 48 are able to be reduced.

### Embodimemt 5

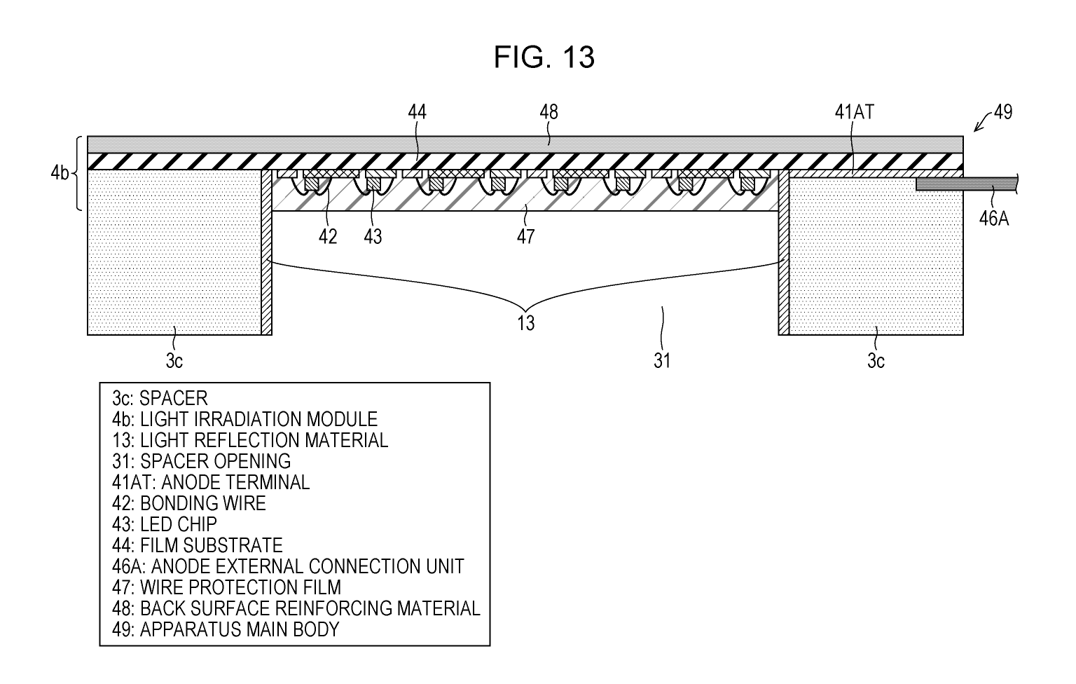

Embodiment 5 of the invention will be described as follows with reference to FIG. 13. Note that, for convenience of description, a member having the same function as that of the member described in the foregoing embodiments will be given the same reference sign and description thereof will be omitted. FIG. 13 is a sectional view of an apparatus main body of a phototherapy apparatus 100d according to Embodiment 5.

The phototherapy apparatus 100d according to the present embodiment is the same as that of Embodiment 1 excluding that an apparatus main body 49 is provided in place of the apparatus main body 9 (refer to FIG. 1). In the light irradiation module 4 of the apparatus main body 9 illustrated in FIG. 4, the LED chips 43 are arranged up to an outside of the spacer opening 31. On the other hand, in the present embodiment, the LED chips 43 are arranged only on an inside of the spacer opening 31 in a light irradiation module 4b of the apparatus main body 49. Note that, though not illustrated in FIG. 13, a configuration may be such that wires 41CT, 41AT, 41CV, 41AV, 41C, 41A, and 45, the wire protection film 47, and the like are provided on the outside of the spacer opening 31 for reinforcement.

The present embodiment has a configuration in which a spacer 3c is constituted by a light absorbing material, and thus the light shielding material 5 is not provided.

Further, a light reflection material 13 is arranged on an inner wall of the spacer opening 31. The light reflection material 13 causes light emitted from the LED chips 43 that are on the inside of the spacer opening 31 to be reflected to the inside of the spacer opening 31. By covering the inner wall of the spacer opening 31 with the light reflection material 13, the distribution of light irradiation intensity in the spacer opening 31 is able to be uniformized.

Further, in such a configuration, the sectional shape of the spacer opening 31 is preferably a rectangular shape perpendicular to a front surface of the light irradiation module 4b. A distance between a position where the light reflection material 13 contacts the light irradiation module 4b and each of the LED chips 43 arranged oa an outermost peripheral is preferably about a half of an arranging pitch of the LED chips 43.

Such a sectional shape and a distance are provided to cause light incident on the inner wall of the spacer opening. 31 to be efficiently reflected to an outer edge of the spacer opening 31. Note that, the light reflection material 13 is not essential, but is preferably provided to increase efficiency of light irradiation.

As a modified example of the present embodiment, a configuration in which the front surface of the light irradiation module 4b, which is exposed into the spacer opening 31, is covered by a transparent resin layer is also possible. With the configuration, similarly to Embodiment 4, a transparent resin part in the spacer opening 31 acts as a reinforcing structure. Further, various modification of the light irradiation module 4b may be possible., for example, since deformation of the light irradiation module 4b at the spacer opening 31 is able to be suppressed and prevented without providing the back surface reinforcing material 48.

### Embodiment 6

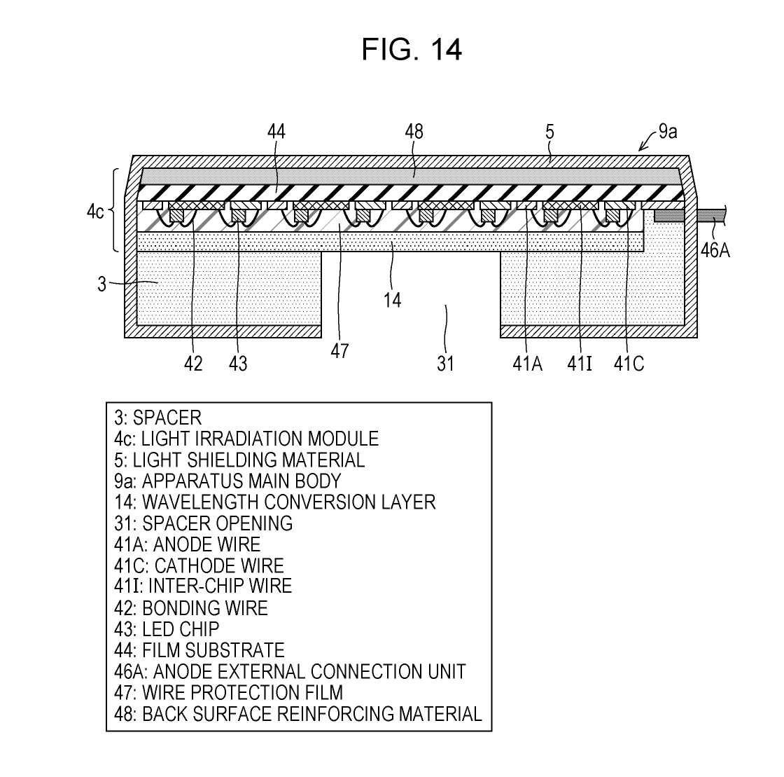

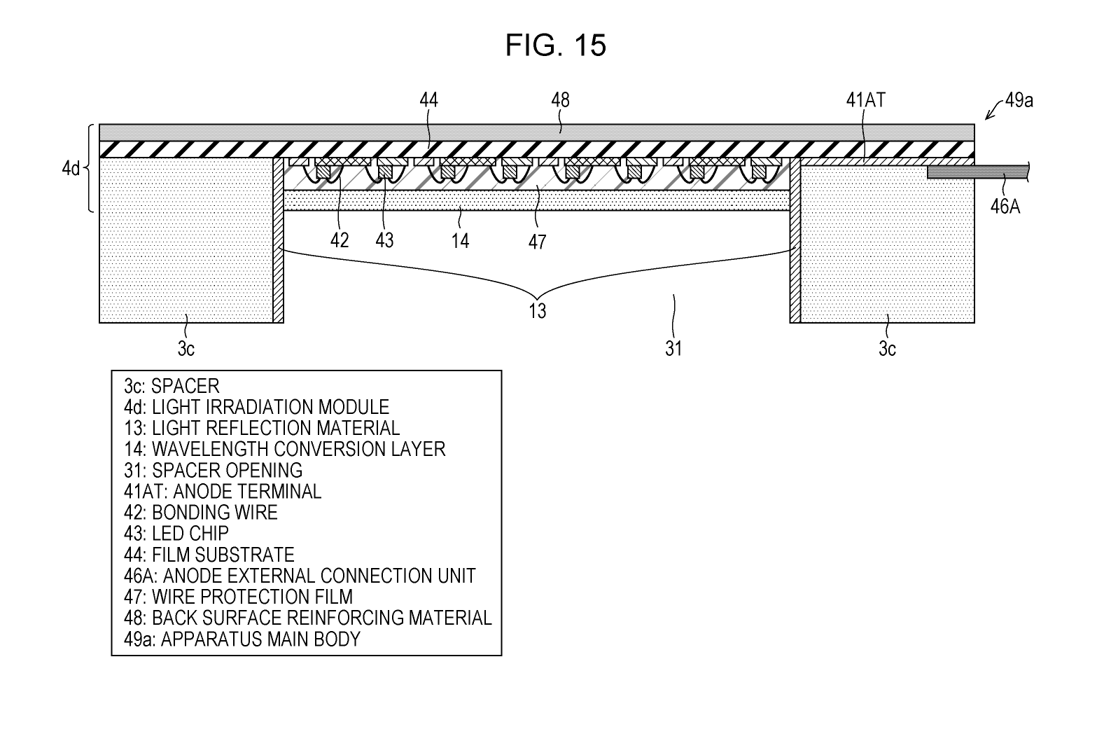

Embodiment 6 of the invention will be described as follows with reference to FIGS. 14 and 15. Note that, for convenience of description, a member having the same function as that of the member described in the foregoing embodiments will be given the same reference sign and description thereof will be omitted. FIG. 14 is a sectional view of an apparatus main body of a phototherapy apparatus 100e according to Embodiment 6.

The phototherapy apparatus 100e according to the present embodiment is the same as the phototherapy apparatus 100 excluding that the light irradiation module 4 is changed to a light irradiation module 4c and the apparatus main body 9 is changed to an apparatus main body 9a as illustrated in FIG. 14 (refer to FIG. 4).

In the light irradiation module 4 of the phototherapy apparatus 100 illustrated in FIG. 4, the affected part 2 is irradiated with light emitted from the LED chips 43 and the light becomes therapeutic light. On the other hand, the light irradiation module 4c of the present embodiment includes a wavelength conversion layer 14. The wavelength conversion layer 14 is able to convert a part of the light emitted by the LED chips 43 into a long wavelength.

For example, an LED chip 43 that emits blue-violet light with a wavelength of about 410 nm is combined with the wavelength conversion layer 14 in which β-type SALON which is a green light-emitting phosphol is kneaded in a transparent resin layer. This makes it possible to generate mixture light of blue-violet light and green light.

Since a ratio of the blue-violet light and the green light is decided on the basis of a content of a phosphor contained in the wavelength conversion layer 14, the phototherapy apparatus 100e that emits mixture light which contains only a little blue-violet light is also able to be achieved. As each of the LED chips 43, a nitride semiconductor LED that emits short-wavelength light such as ultraviolet light, near-ultraviolet light, or blue light is suitable. As a phosphor contained in the wavelength conversion layer 14, various materials and compositions are able to be selected in accordance with a required wavelength range of, for example, blue-green, green, yellow, red, or near-infrared light. For example, a YAG (Yttrium Aluminum Garne) phosphor is usable for a yellow phosphor, a nitride phosphor (CaAlSiN₃ or the like), a fluoride phosphor (NSF (K₂SiF₆:Nln) or the like), or the like is usable for a red phosphor. A material of the wavelength conversion layer 14 is not limited to the phosphor and may be a material such as a quantum dot material. Note that, though the wavelength conversion layer 14 is arranged on the spacer 3 side of the wire protection film 47 in FIG. 14, when a phosphor or a quantum dot material is mixed in the wire protection film 47, a wavelength conversion function is also able to be imparted to the wire protection film 47. Thereby, the wavelength conversion layer 14 is able to be omitted.

As a modified example of the present embodiment, as illustrated in FIG. 15, a configuration obtained by changing the light irradiation module 4h to a light irradiation module 4d in the phototherapy apparatus 100d illustrated in FIG. 13 is also possible. FIG. 15 is a sectional view of an apparatus main body 49a of the modified example of the phototherapy apparatus 100e illustrated in FIG. 14. The light irradiation module 4d includes the wavelength conversion layer 14 and the wavelength conversion layer 14 is arranged on the spacer opening 31 side of the wire protection film 47. A light irradiation module is only required to be a light source that emits therapeutic light in a planar manner in a certain range, like the light irradiation module 4c or 4d.

### Embodiment 7

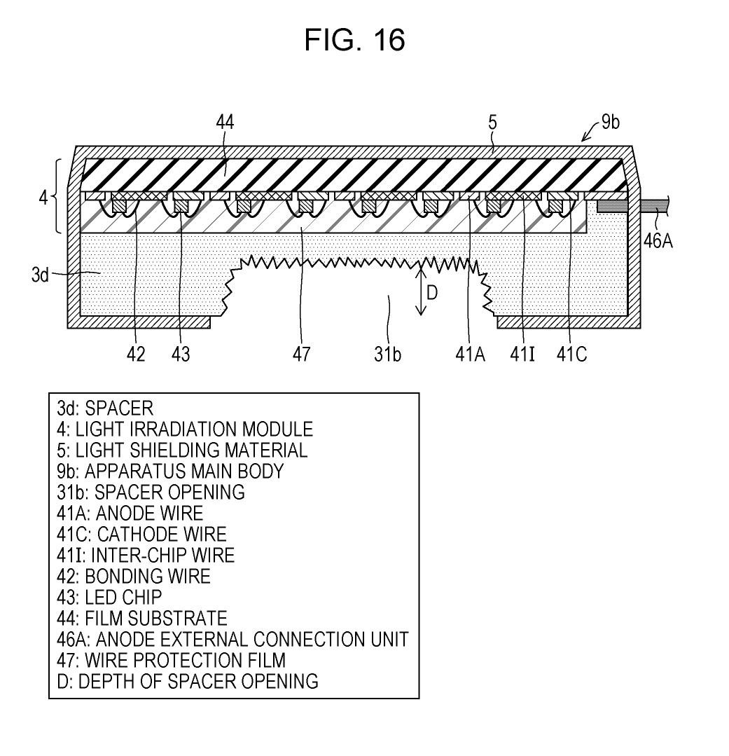

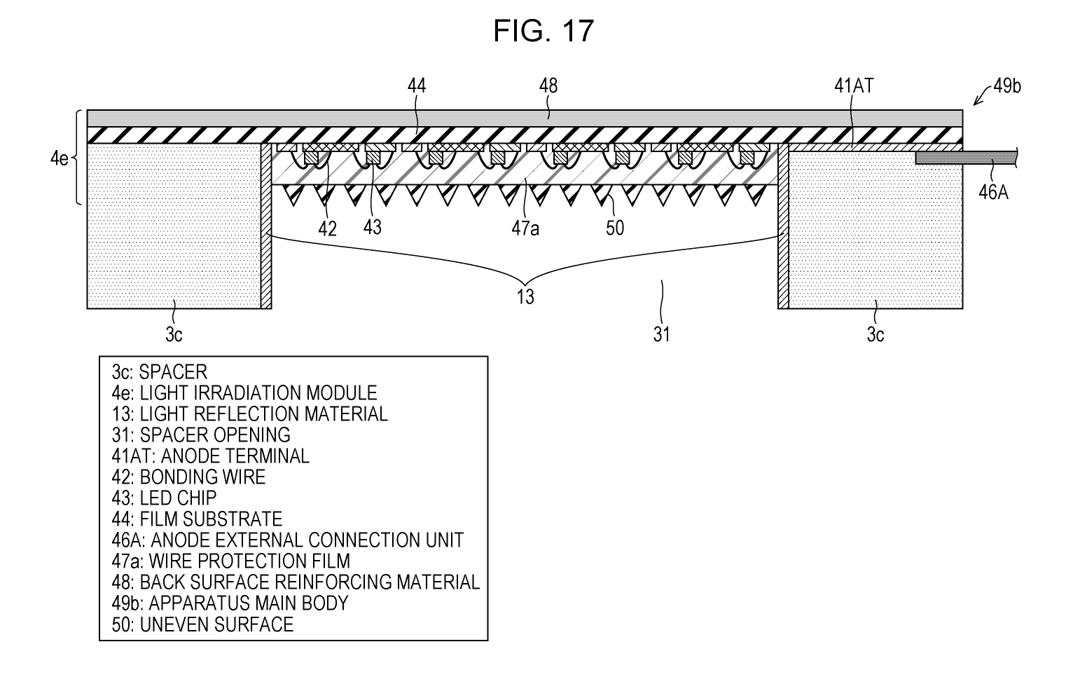

Embodiment 7 of the invention will be described as follows with reference to FIGS. 16 and 17. Note that, for convenience of description, a member having the same function as that of the member described in the foregoing embodiments will be given the same reference sign and description thereof will be omitted. FIG. 16 is a sectional view of an apparatus main body of a phototherapy apparatus 100f according to Embodiment 7.

The phototherapy apparatus 100f according to the present embodiment is the same as the phototherapy apparatus 100c excluding that the spacer 3b is changed to a spacer 3d as illustrated in FIG. 16 (refer to FIG. 12). While an inner surface of the spacer opening 31b is flat in the spacer 3b of the phototherapy apparatus 100c, an inner surface of the spacer opening 31b has an uneven shape in the spacer 3d. This is because therapeutic light is more efficiently extracted to the spacer opening 31b. The inner surface of the spacer opening 31b means a bottom surface and a side surface of the spacer opening 31b.

A process where light is output from inside of the spacer 3b to an air layer when the inner surface of the spacer opening 31b is flat is considered. In the process, when an incident angle of the light from the inside of the spacer 3b to the air layer is larger than a critical angle which is decided by refractive indices of the air layer and a material of the spacer 3b, total reflection occurs. Thereby, efficiency of extracting the light from the spacer 3b is reduced and power efficiency is also reduced. The air layer is a layer that is in the spacer opening 31b.

In FIG. 16, an uneven shape of the spacer 3d is formed randomly, but may be formed in a regular pattern. A diameter of a bottom part of a convex part is preferably one μm to several tens of μm and a height of the convex part is also preferably one μm to several tens of μm. The uneven shape of the spacer 3d is preferably a circular shape, an elliptical shape, a triangular shape, a hexagonal shape, an octagonal shape, or the like, and a sectional shape of the unevenness of the spacer 3d is preferably a semicircular shape, a semielliptical shape, a pyramid shape, a conic shape, or the like.

In molding processing of the spacer 3d with use of a die, when a surface of the die, which corresponds to the inner surface of the spacer opening 31b, is formed in an uneven shape, the uneven shape as illustrated in FIG. 16 is able to be formed. When the uneven shape in which the diameter of the bottom part and the height of the convex part is about 5 μm to 20 μm is formed in the spacer 3d, an output of light is able to be improved by 5% to 10%. Since the output of light is improved, a light irradiation time is able to be shortened and a treatment time is able to be shortened.

Note that, thoughthe uneven shape is provided on the entire inner surface of the spacer opening 31b in FIG. 16, an almost similar effect is achieved also when the uneven shape is provided only on the bottom surface (surface parallel to a bottom surface of the phototherapy apparatus 100f) of the spacer opening 31b in consideration of restriction of manufacturing. The entire inner surface includes a parallel surface and a perpendicular surface to the bottom surface of the phototherapy apparatus 100f.

As described above, by providing the uneven shape on the inner surface of the spacer opening 31b like the uneven shape of the spacer 3d, efficiency of extracting light is able to be improved and a treatment time is able to be shortened.

As a modified example of the present embodiment, as illustrated in FIG. 17, a configuration obtained when a front surface of the wire protection film 47 of the apparatus main body 49 is formed in an uneven shape in the phototherapy apparatus 100d illustrated in FIG. 13 is also possible. FIG. 17 is a sectional view of an apparatus main body 49b of the modified example of the phototherapy apparatus 100f illustrated in FIG. 16. In the phototherapy apparatus 100d illustrated in FIG. 13, since the spacer 3c does not have a surface (surface parallel to a bottom surface of the phototherapy apparatus 100d) from which light is emitted, a wire protection film 47a has an uneven surface 50 on the front surface thereof. Note that, the uneven surface 50 may be made from a transparent material different from that of the wire protection film. An uneven shape of the uneven surface 50 has a regular pattern, and a minimum unit thereof is an equilateral triangle one side of which has a length of 5 μm, and each convex part has an almost conic shape. The minimum unit is specifically an equilateral triangle formed by being surrounded by lines connecting vertexes of convex parts of the uneven surface 50, which are most proximate to each other, when the uneven surface 50 is seen from a surface side thereof. A diameter of a bottom surface of a cone of each of the convex parts is 4 μm and a height of the cone of each of the convex parts is also 4 μm. By providing the uneven shape on the bottom surface (surface parallel to the bottom surface of the phototherapy apparatus 100f) of the spacer opening 31 as in the uneven surface 50 of the wire protection film 47a, efficiency of extract Ing tight is able to be improved and a treatment time is able to be shortened.

Note that, the effect of shortening of the treatment time or the like is achieved by providing the uneven shape on the spacer 3d or the wire protection film 47a, only when there is an asir layer between the affected part 2 and the inner surface of the spacer opening 31. The aforementioned effect, is an effect specific to a phototherapy apparatus targeted for condition of disease in which direct contact with the affect part 2 is not preferable.

### CONCLUSION

A phototherapy apparatus 100, 100a, 100b, 100c, 100f according to an aspect 1 of the invention includes: a light irradiation module 4, 4A that radiates light for treatment; and a spacer 3, 3a, 3b, 3d that is disposed on a light output side of the light irradiation module 4, 4A and contacts a surface of a body 1 to keep a distance between the light irradiation module 4, 4A and a part (affected part 2) to be treated on the surface of the body 1 constant, in which an opening. (spacer opening 31, 31a, 31b) that avoids contact of the part to be treated and the spacer 3, 3a, 3b, is formed on a surface of the spacer 3, 3a, 3b, which contacts the surface of the body 1.

According to the aforementioned configuration, in the phototherapy apparatus, by forming the opening in the spacer, the part to be treated is not allowed to directly contact the spacer, thus making possible to reduce burden on the part to be treated.

The phototherapy apparatus 100a, 100b, 100c according to an aspect 2 of the invention further has a configuration in which the spacer 3b has a light-transmitting property to transmit the light, and the opening (spacer opening 31b) is a concave part formed on the surface contacting the surface of the body 1.

According to the aforementioned configuration, the light irradiation module 4 is able to be prevented from being deformed at the opening. In a case where the spacer 3b has a function of uniformizing light output from the light irradiation module 4, there is also an effect that light radiated to the part to be treated is able to be uniformized.

The phototherapy apparatus 100, 100a, 100b, 100c according to an aspect 3 of the invention further has a configuration in which the light irradiation module 4, 4A has a reinforcing structure to prevent deformation by the opening (spacer opening 31, 31a).

According to the aforementioned configuration, strength of the light irradiation module 4, 4A is able to be increased, so that local sinking of the light irradiation module 4, 4A at the opening is able to be prevented and an excessive increase of light irradiation intensity in a center of the opening is able to be prevented, compared with that in a peripheral part of the spacer.

The phototherapy apparatus 100a according to an aspect 4 of the invention further has a configuration in which the spacer 3a is formed in a lattice pattern, and the opening (spacer opening 31a) is formed by cutting a lattice part.

According to the aforementioned configuration, a partition part of the lattice pattern is narrow, and is thus easily cut by a tool such as scissors or nippers and a size of the open-ing is able to be minimized, so that an area of a normal part subjected to light irradiation in skin is able to be reduced to a minimum. Since the size of the opening is able to be minimized, deformation of the light irradiation module at the opening is able to be suppressed.

The phototherapy apparatus 100, 100a, 100b, 100c according to an aspect 5 of the invention further has a configuration in which the spacer 3, 3a, 3b has a light-transmitting property to transmit the light, the phototherapy apparatus includes a light shielding unit (light shielding material 5) that shields the light, and the light shielding unit covers the light irradiation module 4, 4A and the spacer 3, 3a, 3b other than the opening (spacer opening 31, 31a, 31b) or other than the opening and an edge thereof.

According to the aforementioned configuration, by providing the light shielding unit, it is possible to reduce unnecessary light irradiation to a normal part of skin around the part to be treated when light is radiated from the light irradiation module 4, 4A, and reduce a side effect on the normal part of the skin. Since powerful light emitted from the light irradiation module 4, 4A during treatment is able to be prevented from being leaked to outside from the phototherapy apparatus 100, 100a, 100b, 100c, a patient or a medical practitioner does not need to wear special protective equipment. It is possible to reduce unnecessary light irradiation to a normal part of skin and reduce a side effect on the normal part of the skin.

The phototherapy apparatus 100, 100a, 100b, 100c according to an aspect 6 of the invention further has a configuration in which the spacer 3, 3a, 3b has a light-transmitting property to transmit the light, the light irradiation module includes a film substrate 44 having flexibility and a plurality of light emitting elements (LED chips 43) mounted on the film substrate 44, and the plurality of light emitting elements are arranged also on an outside of a part corresponding to the opening (spacer opening 31, 31a, 31b).

According to the aforementioned configuration, since light outside the opening also reaches an inside of the opening via the spacer, light intensity at an end of the opening, at which light intensity is easily reduced compared to that in a center part of the opening, is able to be increased and uniform light irradiation intensity is able to be achieved inside the opening.

The phototherapy apparatus 100, 100a, 100b, 100c according to an aspect 7 of the invention further has a configuration in which at least a surface of the light shielding unit (light shielding material 5), which contacts the spacer, is constituted by a material by which the light is reflected.

According to the aforementioned configuration, with the reflection by the light shielding unit, the light is able to reach a target part, though only a little, thus making it possible to increase irradiation efficiency to the target part.

The phototherapy apparatus 100f according to an aspect 8 of the invention further has a configuration in which an inner surface of the opening (spacer opening 31, 31b) has an uneven shape.

According to the aforementioned configuration, since the inner surface of the opening has the uneven shape, efficiency of extracting light is able to be improved and a treatment time is able to be shortened.

The phototherapy apparatus 100f according to an aspect 9 of the invention further has a configuration in which the opening (spacer opening 31b) is a concave part formed in the spacer 3d.

### REFERENCE SIGNS LIST

1 body

2 affected part (part to be treated)

3, 3a, 3b, 3c, 3d spacer

4, 4b, 4c, 4d light irradiation module

5, 5a light shielding. material (light shielding unit)

6 main body fixing material (fixing member)

7 cooling material

8 cooling material fixing. material

9, 49, 49a, 49b apparatus main body

11 main body fixing material/skin adhesive portion

12 cooling material fixing material/skin adhesive portion

13 light reflection material

14 wavelength conversion layer

20 power source unit

21 power source control unit

22 current supply unit

23 signal line

31, 31a, 31b spacer opening (opening)

41A, 51A anode wire (first wire)

41AT anode terminal

41AV anode vertical wire

41C, 51C cathode wire (first wire)

41CT cathode terminal

41CV cathode vertical wire

41I inter-chip wire

42 bonding wire

43 LED chip

44, 54 film substrate

45 dummy pattern

46A anode external connection soft

46C cathode external connection unit

47, 47a wire protection film

48 back surface reinforcing material (reinforcing member)

57 back-side anode wire (second wire)

58 back-side cathode wire (second wire)

59 anode wire through wire

60 cathode wire through wire

100, 100a, 100b, 100c, 100d, 100e, 100f phototherapy apparatus

1. A phototherapy apparatus comprising:
a light irradiation module that radiates light for treatment; and
a spacer that is disposed on a light output side of the light irradiation module and contacts a surface of a body to keep a distance between the light irradiation module and a part to be treated on the surface of the body constant, wherein
an opening that avoids contact of the part to be treated and the spacer is formed on a surface of the spacer, which contacts the surface of the body,
the spacer has a light-transmitting property to transmit the light, and
the opening is a concave part formed on the surface contacting the surface of the body.

2. (canceled)

3. The phototherapy apparatus according to claim 1, wherein the light irradiation module has a reinforcing structure to prevent deformation by the opening.

4. The phototherapy apparatus according to claim 1, wherein
the spacer is formed in a lattice pattern, and
the opening is formed by cutting a lattice part.

5. The phototherapy apparatus according to claim 1, wherein
further comprising:
a light shielding unit that shields the light, wherein
the light shielding unit covers the light irradiation module and the spacer other than the opening or other than the opening and an edge thereof.

6. The phototherapy apparatus according to claim 1, wherein
the light irradiation module includes a film substrate having flexibility and a plurality of light emitting elements mounted on the film substrate, and
the plurality of light emitting elements are arranged also on an outside of a part corresponding to the opening.

7. The phototherapy apparatus according to claim 5, wherein at least a surface of the light shielding unit, which contacts the spacer, is constituted by a material by which the light is reflected.

8. The phototherapy apparatus according to claim 1, wherein an inner surface of the opening has an uneven shape.

9. (canceled)

10. A phototherapy apparatus comprising:
a light irradiation module that radiates light for treatment; and
a spacer that is disposed on a light output side of the light irradiation module and contacts a surface of a body to keep a distance between the light irradiation module and a part to be treated on the surface of the body constant, Wherein
an opening that avoids contact of the part to be treated and the spacer is formed on a surface of the spacer, which contacts the surface of the body,
the spacer has a light-transmitting property to transmit the light,
the light irradiation module includes a film substrate having flexibility and a plurality of light emitting elements mounted on the film substrate, and
the plurality of light emitting elements are arranged also on an outside of a part corresponding to the opening.

11. The phototherapy apparatus according to claim 10, wherein the light irradiation module has a reinforcing structure to prevent deformation by the opening.

12. The phototherapy apparatus according to claim 10, wherein
the spacer is formed in a lattice pattern, and
the opening is formed by cutting a lattice part.

13. The phototherapy apparatus according to claim 10, further comprising:
a light shielding unit that shields the light, wherein
the light shielding unit covers the light irradiation module and the spacer other than the opening or other than the opening and an edge thereof.

14. The phototherapy apparatus according to claim 13, wherein at least a surface of the light shielding unit, which contacts the spacer, is constituted by a material by which the light is reflected.

15. The phototherapy apparatus according to claim 10, wherein an inner surface of the opening has an uneven shape.

16. The phototherapy apparatus according to claim 15, wherein the opening is a concave part formed in the spacer.

17. A phototherapy apparatus comprising:
a light irradiation module that radiates light for treatment; and
a spacer that is disposed on a light output side of the light irradiation module and contacts a surface of a body to keep a distance between the light irradiation module and a part to be treated on the surface of the body constant, wherein
an opening that avoids contact of the part to be treated and the spacer is formed on a surface of the spacer, which contacts the surface of the body, and
an inner surface of the opening has an uneven shape.

18. The phototherapy apparatus according to claim 7 wherein the opening is a concave part formed in the spacer.
